(19) United States
(12) Patent Application Publication
(10) Pub. No.: US 2023/0183801 A1
(43) Pub. Date: Jun. 15, 2023

(54) SENESCENT CELL DETERMINATION SYSTEM

(71) Applicant: TERUMO KABUSHIKI KAISHA, Tokyo (JP)

(72) Inventors: Eri NOGUCHI, Ashigarakami-gun (JP); Masaki MATSUMURA, Ashigarakami-gun (JP)

(73) Assignee: TERUMO KABUSHIKI KAISHA, Tokyo (JP)

(21) Appl. No.: 18/166,260

(22) Filed: Feb. 8, 2023

Related U.S. Application Data
(63) Continuation of application No. PCT/JP2021/036035, filed on Sep. 30, 2021.

(30) Foreign Application Priority Data
Sep. 30, 2020 (JP) 2020-165843

(51) Int. Cl. C12Q 1/6876; A61K 35/12

(52) U.S. Cl. CPC C12Q 1/6876; A61K 35/12

(57) ABSTRACT

A system that non-destructively and quickly determines a degree of senescence of cells to be cultured by utilizing the discovery that when cells are adhered to a surface of a culture substrate in a culture medium, the degree of cytoplasm spreading on the culture substrate is indicative of the degree of senescence. The degree of the spreading can be detected based on a result of brightness, reflectance, or image processing, and the degree of senescence is determined. It is also possible to determine the degree of senescence from a rate of the spread.

### CROSS-REFERENCES TO RELATED APPLICATIONS

This application is a continuation of International Patent Application No. PCT/JP2021/036035 filed on Sep. 30, 2021, which claims priority from Japanese Patent Application No. 2020-165843 filed on Sep. 30, 2020, the entire content of both of which is incorporated herein by reference.

### TECHNOLOGICAL FIELD

The present disclosure generally relates to a system that determines senescent cells in a cell product for culturing a target cell.

### BACKGROUND DISCUSSION

In recent years, attempts have been made to transplant various cells for repair of damaged tissues and the like. For example, attempts have been made to use fetal cardiomyocytes, myoblast cells, mesenchymal stem cells, cardiac stem cells, ES cells, iPS cells, and the like for repair of myocardial tissue damaged by ischemic heart disease such as angina pectoris and myocardial infarction (Haraguchi et al., Stem Cells Transl Med. 2012 February; 1(2): 136-41).

As a part of such attempts, a cell structure formed using a scaffold and a sheet-shaped cell culture in which cells are formed in a sheet shape have been developed (Sawa et al., Surg Today. 2012 January; 42(2): 181-4).

For the application of the sheet-shaped cell culture to treatment, studies have been made on the use of a cultured epidermal sheet for skin damage caused by burns or the like, the use of a corneal epithelial sheet-shaped cell culture for corneal damage, the use of an oral mucosa sheet-shaped cell culture for endoscopic resection of esophageal cancer, and the like, and some of them have entered the stage of clinical application.

Myoblast cells used for such treatment are usually obtained by separating CD 56 positive cells such as myoblast cells and muscle satellite cells from skeletal muscle tissue to be transplanted. As a measure to increase a ratio of the CD 56 positive cells contained in cells separated from the skeletal muscle tissue, for example, a method is known that includes a first process involving immersing skeletal muscle tissue in a first protease solution for a prescribed time and then discarding the obtained first enzyme-treated solution and a second process involving immersing the skeletal muscle tissue resulting from the first process in a second protease solution for a prescribed time and then recovering the cells contained in the second enzyme-treated solution (JP 2007-89442 A).

In a case where myoblast cells are used as, for example, a cell product as a material for producing a sheet-shaped cell culture, the number of cells is increased by culturing the cells, but when the cells are aged, the proliferation is stopped in the middle, and a certain number of cells cannot be obtained.

In order to avoid such a risk, it is necessary to determine a degree of senescence of cells to be subjected to culture, and determine whether or not the content of senescent cells in a cell-containing liquid to be determined is within an acceptable range.

Conventionally, in order to know a degree of senescence of cells for cell culture, a part of a liquid containing the cells has been sampled and analyzed, and thus it cannot be said to be non-destructive inspection, and a part of the cells has been lost for inspection. Furthermore, the analysis takes time.

As a technique for determining a degree of activity of division while cells remain alive, there has been known a technique in which microscopic observation is performed using a dye that does not kill cells even when bound (JP 08-136536 A).

However, this observation method is performed by a human by microscopic observation, and cannot automatically discriminate a large amount of target cells, for example. Furthermore, there are problems that it takes a large amount of time to make a determination and that the determination varies in the case of visual observation by a human.

### SUMMARY

Conventionally, in a case where cultured cells are to be supplied as a cell product, if senescent cells whose proliferation stops in the middle when the cell product is further cultured are included, the cells are ineligible as a cell product. Therefore, it is necessary to determine a degree of senescence of the cell product. If the cells can be determined non-destructively, a risk of supplying ineligible products can be avoided without loss of cells. Therefore, such determination has been desired.

Furthermore, it is desirable that the non-destructive cellular senescence determination can be performed quickly and easily. Moreover, highly accurate determination without variation has been desired for the determination of the degree of senescence.

As a result of intensive studies to solve the above-described problems, the present inventors have found that when cells to be cultured adhere to a culture substrate, an area of cytoplasm of the cells expands, but the expansion is greatly different between senescent cells and non-senescent cells, and the expansion is different between senescent cells and non-senescent cells even in a state after cell proliferation.

Furthermore, it has been found that the expansion of the area of the cytoplasm can be detected non-destructively by the reflectance of light, the brightness of cells, or captured image information. As a result of further research based on such findings, the present inventors have found that a degree of senescence of a target cell to be cultured can be detected quickly by a non-destructive method, and inspection of the quality of a cell product can be automated.

Furthermore, since it has also been found that the speed at which the cytoplasm area expands after adhesion to a culture substrate, and the speed of the expansion at the time of cell proliferation, that is, the time change rate of the area, also vary depending on the degree of senescence of the cultured cells, the degree of senescence of the cultured cells can also be determined by detecting an area change rate per unit time, and the present disclosure also includes this.

That is, the present disclosure relates to the following.

[1]A system that determines senescence of a target cell, the system including: a detection unit that detects a change in an area of cytoplasm of the target cell on a culture substrate; and a determination unit that determines the senescence of the cell from a detection result of the detection unit.

[2] The system according to [1], in which the determination unit determines the senescence from the change in the area after adhesion of the target cell to the culture substrate.

[3] The system according to [1] or [2], in which the detection unit detects the change in the area of the cytoplasm from brightness, reflectance, or image information of the cell.

[4] The system according to any one of [1] to [3], in which the change in the area of the cytoplasm is detected by a time change rate of the area.

[5] The system according to any one of [1] to [4], in which the determination unit determines a difference in the change in the area of the cytoplasm dependent on a degree of senescence by causing the determination unit to learn an actually measured degree of senescence.

[6] The system according to any one of [1] to [5], wherein the target cell is a myoblast.

Another aspect of the present disclosure relates to a method for determining senescence of a target cell, the method including: detecting a change in an area of cytoplasm of the target cell on a culture substrate; and determining senescence of the target cell on the culture substrate with a determination unit.

According to the present disclosure, since the degree of senescence of a target cell can be quickly determined in a non-destructive manner, it is possible to quickly determine the quality of a cell product without losing cells. Furthermore, since the determination can be performed using a detector or an image processing unit using light in a non-destructive and non-contact manner, automatic determination without manual intervention becomes possible, a possibility of occurrence of variations in determination is greatly reduced, and objectivity and stabilization of determination criteria can also be achieved.

### BRIEF DESCRIPTION OF THE DRAWINGS

FIG. 1 depicts a diagram of an exemplary embodiment of a system for determining a spread of cytoplasm.

FIG. 2 illustrates an example of components of a system that determines senescent cells in a cell product for culturing a target cell.

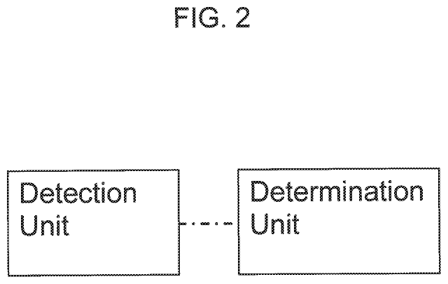

### DETAILED DESCRIPTION

Unless otherwise defined herein, all technical and scientific terms used herein have the same meaning as commonly understood by one of ordinary skill in the art. All patents, applications and other publications and information referenced herein are hereby incorporated by reference in their entirety.

In the present disclosure, the “cell” can include any cell separated from a living body-derived tissue. Non-limiting examples thereof include cardiomyocytes, fibroblast cells, epithelial cells, endothelial cells, hepatocytes, pancreatic cells, renal cells, adrenal cells, periodontal ligament cells, gingival cells, periosteal cells, skin cells, synoviocytes, chondrocytes, and the like, and stem cells (for example, myoblasts (for example, myoblast cells) (Myoblasts include satellite myocytes), mesenchymal stem cells (for example, those derived from bone marrow, adipose tissue, peripheral blood, skin, hair roots, muscle tissue, endometrium, placenta, cord blood, and the like), tissue stem cells such as cardiac stem cells, embryonic stem cells, etc.). The cell in the present disclosure may be a cell that is located between a plurality of membranes that are in planar contact so as to be surrounded by the membranes such that the cell is in direct contact with the plurality of membranes or adjacent to the plurality of membranes. In the present disclosure, the cells preferably include CD 56 positive cells in skeletal muscle tissue such as myoblast cells, or mesenchymal stem cells derived from bone marrow, adipose tissue, and peripheral blood.

In the present disclosure, “cell senescence” means that the number of divisions of a cell has an upper limit to some extent, and when the number of divisions increases to a certain extent, no further division is performed. That is, the cell senescence means that a state in which cells stop dividing and cannot proliferate is irreversibly caused.

Therefore, it is considered that, when the cells are proliferated, in a case where a large amount of senescent cells are contained, the proliferation thereof is reduced.

In the present disclosure, “brightness” refers to a degree of brightness of a cell when the cell is observed, and since a cytoplasm portion in the field of view is brighter than a ground portion, an area of the cytoplasm in the field of view is obtained by measuring the brightness of the entire field of view.

The “reflectance” is a ratio of the intensity of reflected light to the intensity of irradiation light when the cell is irradiated with light.

These can be quantitatively detected by measuring light intensity with a photocell. That is, the intensity of reflected light and the reflectance (ratio of the intensity of reflected light) can be quantitatively detected by measuring light intensity with a photocell.

The higher the degree of senescence, the larger the amount of expansion of cytoplasm when adhered to a substrate. Typical young myoblasts spread to about 200% when adhered to the substrate, compared to before adhesion. But the senescent cells spread to about 300% when adhered to the substrate, compared to before adhesion

Therefore, as the degree of senescence, the cytoplasm area can be detected by a well-known image recognition method by image brightness, light reflectance, or image processing corresponding to the degree of spread of the cytoplasm at the time of adhesion.

Furthermore, since the spread of the cytoplasm after cell proliferation is also larger as the degree of senescence is higher, the degree of senescence can also be determined from the spread of the cytoplasm of the cells after proliferation on the substrate.

That is, the degree of senescence can be determined from the spread of the cytoplasm after cell proliferation by detecting the cytoplasm area by a well-known image recognition method by image brightness, light reflectance, or image processing.

Detecting an area change after the time of adhesion of the present disclosure also includes detecting a cytoplasm area after cell proliferation.

Moreover, it is also possible to determine the degree of senescence by the above-described index, that is, the time change rate of the area value obtained by the brightness, the reflectance, or the image processing.

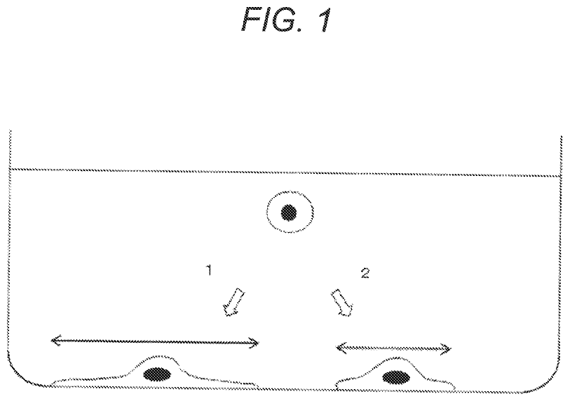

As illustrated in FIG. 1, senescent cells (1 of FIG. 1) spread more cytoplasm than young cells (2 of FIG. 1), but spread faster by the amount of no elasticity in cytoplasm. Therefore, it is also possible to determine the degree of senescence from the rate of change in the index described above.

Since the brightness, the reflectance, or the area of the cytoplasm recognized by the image can be automatically detected without human intervention, the senescence state determination can be automated.

When a determination threshold value is set to the measurement value as described above as a criterion for the senescence state, an automatic cell product quality determination system can be constructed.

Therefore, in a situation such as pre-shipment inspection of a cell product, it is possible to automatically determine the grade of the degree of senescence of the product and efficiently classify the quality of the cell product.

Furthermore, this determination threshold value can be calibrated by an actual measurement result obtained by actually sampling and analyzing a determination result at the time of system operation, and as the determination threshold value is operated, it becomes possible to accumulate results of the determination result, and it becomes possible to improve accuracy of the system.

The detailed description above describes a system and method for determining that senescent cells in a cell product for culturing a target cell representing examples of the inventive system and method disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A method for determining senescence of a target cell, the method comprising:
detecting a change in an area of cytoplasm of the target cell on a culture substrate; and
determining senescence of the target cell on the culture substrate using the detected change in area.

2. The method according to claim 1, wherein the detecting of the change in the area of cytoplasm of the target cell on the culture substrate includes detecting brightness of the target cell or reflectance of the target cell.

3. The method according to claim 1, wherein the change in the area of the cytoplasm is detected by a time change rate of the area.

4. The method according to claim 1, wherein the target cell is a myoblast.

5. The method according to claim 1, wherein the target cell is a cardiomyocyte, fibroblast cell, epithelial cell, endothelial cell, hepatocyte, pancreatic cell, renal cell, adrenal cell, periodontal ligament cell, gingival cell, periosteal cell, skin cell, synoviocyte, chondrocyte or a stem cell.

6. The method according to claim 1, wherein the target cell is derived from a skeletal muscle tissue containing CD 56 positive cells.

7. The method according to claim 1, wherein the detecting of the change in the area of cytoplasm of the target cell on the culture substrate includes detecting the change in the area of cytoplasm of the target cell after cell proliferation.

8. A system that determines senescence of a target cell, the system comprising:
a detection unit that detects a change in an area of cytoplasm of the target cell on a culture substrate; and
a determination unit that determines the senescence of the cell from a detection result of the detection unit.

9. The system according to claim 8, wherein the determination unit determines the senescence from the change in the area after adhesion of the target cell to the culture substrate.

10. The system according to claim 8, wherein the detection unit detects the change in the area of the cytoplasm from brightness, reflectance, or image information of the cell.

11. The system according to claim 10, wherein the change in the area of the cytoplasm is detected by a time change rate of the area.

12. The system according to claim 8, wherein the determination unit determines a difference in the change in the area of the cytoplasm dependent on a degree of senescence by causing the determination unit to learn an actually measured degree of senescence.

13. The system according to claim 8, wherein the target cell is a myoblast.
